(19) United States
(12) Patent Application Publication
(10) Pub. No.: US 2022/0025467 A1
(43) Pub. Date: Jan. 27, 2022

(54) KCNQ1OT1, A NEW BIOMARKER IN PERIPHERAL BLOOD FOR NON-INVASIVE DETECTION OF GASTRIC CANCER (GC)

(71) Applicant: PONTIFICIA UNIVERSIDAD CATOLICA DE CHILE, Santiago (CL)

(72) Inventors: Alejandro CORVALÁN, Santiago (CL); Wilda OLIVARES, Santiago (CL); Pablo SANTORO, Santiago (CL)

(21) Appl. No.: 17/309,643

(22) PCT Filed: Dec. 14, 2018
(86) PCT No.: PCT/CL2018/050126

§ 371 Date: Jun. 11, 2021

(51) Int. Cl. C12Q 1/6886; C12Q 1/686

(52) U.S. Cl. CPC C12Q 1/6886; C12Q 1/686

(57) ABSTRACT

The invention relates to a non-invasive method for detecting gastric cancer based on the detection of a new molecular marker in blood. In particular, via the detection of the long non-coding RNA (LncRNA) KCNQ1OT1 in peripheral blood, wherein an increase in the expression of this LncRNA correlates with gastric cancer or the precursor lesions thereof (OLGA stage III and IV).

### FIELD OF THE INVENTION

The invention aims at a method for non-invasive detection of gastric cancer based on the detection of a molecular marker in blood. In particular, through the detection in peripheral blood of the long non-coding RNA (IncRNA) KCNQ1OT1 where an increase in the expression of this IncRNA is correlated with gastric cancer or its precursor lesions (OLGA III AND IV).

### BACKGROUND

Gastric cancer is the fifth most common cancer and the third leading cause of death in the world. Despite the advances in its treatment, the prognosis is negative since it is frequently detected in advanced stages. When the disease is confined to the mucosa and submucosa layers of the stomach (early stages), the survival rate is 95% at 5 years. In contrast, when it extends to the muscularis propria or serous layers (advanced stages), patient survival drops substantially to 10-20% at 5 years.

The diagnosis of gastric cancer in early stages is difficult, since the majority of cases are asymptomatic until a very advanced stage.

There are several biomarkers that are detected directly in non-invasive samples to diagnose this disease in an asymptomatic population. The best known is the marker of gastric atrophy (a precursor lesion of gastric cancer) called pepsinogen I/II. Gastric atrophy has a risk of developing gastric cancer around 0.1%, which implies that these patients will require special surveillance with invasive methods such as radiology and endoscopy.

On the other hand, gastric atrophy, in addition to being the precursor lesion of gastric cancer, is a lesion associated with aging. Therefore, its predictive value is lost as the age of the population under evaluation increases.

Until now, the most widely used tool for its detection is endoscopy associated with obtaining and analyzing stomach biopsies. Within this procedure is the Sydney protocol, which determines the taking of 5 biopsies from specific places in the stomach (2 in the antrum and 2 in the body and one in the angle). The results of these biopsies are analyzed according to the OLGA system (from its acronym in English Operative Link for Gastritis Assessment), which systematizes the results in order to classify the patients into 5 groups, regarding the risk of suffering from gastric cancer, where OLGA 0 is the lowest risk and OLGA IV the highest risk. This method is highly invasive and expensive, since it requires the participation of 2 highly qualified physicians such as the endoscopist and the pathologist. And the patient must endure the discomfort of endoscopy and biopsies.

The need for early diagnostic methods for this disease persists in the current technique and to reduce the high mortality rate associated with gastric cancers detected in advanced stages of the disease. It is necessary to look for new methods of early diagnosis, which are applicable to the asymptomatic population, in a fast, non-invasive, effective and low-cost way and where biopsies are performed in the at-risk group, optimizing human and economic resources, for this complex diagnostic technique.

The invention provides a new early gastric cancer biomarker, which can be studied with non-invasive methods, such as a peripheral blood sample. Specifically, the invention aims at the detection in plasma of the long non-coding RNA (IncRNA) KCNQ1OT1 where an increase in the expression of this IncRNA is correlated with gastric cancer or precursor lesions (OLGA III and IV).

Long non-coding RNAs (IncRNAs) are sequences of more than 200 bp without coding activity, mainly involved in regulatory mechanisms of the genome. Dysregulation of IncRNAs has been associated with the development of several types of cancer. KCNQ1OT1 is an example of IncRNAs, transcribed from the antisense strand of intron 10 of the KCNQ1 gene, located in the 11p15.5 domain of the human genome. The inventors have succeeded in correlating changes in the expression levels of KCNQ1OT1 in peripheral blood or plasma samples from patients with gastric cancer, compared to healthy controls.

### PRIOR ART

In the prior art, KCNQ1OT1 IncRNA in plasma has not been correlated with gastric cancer. The closest document known to the inventors at this time would correspond to the publication of Zhang, Zhiyuan, et al. (“Analysis of IncRNA-Associated ceRNA Network Reveals Potential IncRNA Biomarkers in Human Colon Adenocarcinoma.” Cellular Physiology and Biochemistry 49.5 (2018): 1778-1791). In this publication, Zhang studies the correlation of different IncRNAs as potential biomarkers for colon adenocarcinoma, among the IncRNAs studied is KCNQ10T1. Zhang indicates that the correlation study of KCNQ10T1 as a biomarker for colon adenocarcinoma is negative. The analyses were performed on colon tissue samples, and not on peripheral blood, as in the present invention.

In conclusion, the state of the art has studied the expression of various IncRNAs in cancer tissue, mainly in colon cancer cells, but the use of IncRNA KCNQ1OT1 as a biomarker of gastric cancer or precursor lesion in the tissue has not been reported, nor in its analysis in the tissue itself (biopsy), much less in peripheral blood or plasma, as is done in the present invention.

### DESCRIPTION OF THE FIGURES

FIG. 1. Expression of KCNQ1OT1 in 3 gastric cancer cell lines. The fold change for gastric cancer cell lines SNU5, SNU1, and Hs746T is shown relative to a normal gastric cell control (GES-1). The results plotted are the ratio between the expression value in the samples to be evaluated and the expression value in the control.

FIG. 2. Expression of KCNQ1OT1 in sets of clinical tissue samples from patients with low-grade (OLGA 0-I, N=5) precursor lesions, high-grade (OLGA III-IV, N=13) precursor lesions, tissues from Gastric Cancer patients (tumor, N=4) and set of gastric cancer cell lines (cDNA lines, N=6). The ΔCt is shown, which corresponds to the difference between the Ct of the gene of interest (KCNQ1OT1) compared to the Ct of a reference gene (Rps13). Ct corresponds to the cycle in which the gene of interest reaches a defined threshold. The higher the Ct, the greater the number of amplification cycles of the gene of interest, therefore, the less quantity of the gene of interest in the evaluated sample.

FIG. 3. Expression of KCNQ1OT1 in clinical plasma samples from 3 patients with low-grade precursor lesions (OLGA 0) and plasma from 4 patients with Gastric Cancer (GC). The (−log Ct) is plotted, which corresponds to the negative logarithm in base 10 of the Ct of the gene of interest. This is because the higher the number of amplification cycles required (Ct) the lower the expression of the RNA evaluated, in this case of KCNQ1OT1.

### DESCRIPTION OF THE INVENTION

The invention aims at a new biomarker and a non-invasive method for the early diagnosis of gastric cancer and precursor lesions, measuring the expression levels of long non-coding RNA (IncRNA) KCNQ1OT1 in peripheral blood, where an increase in the expression of this IncRNA is correlates with gastric cancer or precursor lesions (OLGA III and IV).

The early stages of gastric cancer are associated with lesions within the gastric mucosa, which are called precursor lesions and can be classified using the histological classification OLGA (from its acronym in English, Operative Link for Gastritis Assessment) from OLGA 0, the lowest risk, up to OLGA IV, the highest risk. OLGA III and OLGA IV lesions are considered precursor lesions of gastric cancer.

Surprisingly, the inventors have found that the expression of the IncRNA KCNQ1OT1 is increased in patients with gastric cancer and with precursor lesions classified as OLGA III and OLGA IV, and that this increase is detectable in peripheral blood or plasma. These results are of great importance, since they allow for the correlation of the results of a blood test with the presence or increased probability of developing gastric cancer.

The invention thus presents as a useful tool, by itself or in combination with others, for an early diagnosis of gastric cancer, thus reducing the need to perform biopsies as a first approach for patients with gastric discomfort. The method of the invention allows for it to be a first cut-off point, where only patients with positive results for increased expression of the IncRNA KCNQ1OT1 in blood should undergo more frequent controls and evaluation of the gastric mucosa by biopsies.

As indicated, the IncRNA KCNQ1OT1 is a non-coding RNA transcribed from the antisense strand of intron 10 of the KCNQ1 gene, located in the 11p15.5 domain of the human genome, and is approximately 10 kb long. There are two KCNQ1OT1 sequences reported in Genbank: NR_002728.3 of 91671 bp and NG_016178.2 of 98671 bp. The present invention works with the sequence NR_002728.3. However, both sequences are valid for in silico studies, and can be used to design the primers for the inventive method.

The inventors have determined that the expression of IncRNA KCNQ1OT1 is increased very early in tumors in gastric cancer and in precursor lesions. Based on this result and on the need for a reliable biomarker, quick to quantify at low concentrations from non-invasively isolated samples, and at low cost for people; The method of the invention has been developed, which allows the development of gastric cancer to be detected early, since surprisingly the inventors have established that establishing the increase in the expression of this IncRNA, can be correlated with precursor lesions or development of gastric cancer in people.

In this way, the invention brings to the state of the art, the detection of the IncRNA KCNQ1OT1, as a biomarker of gastric cancer, in samples obtained non-invasively, preferably from peripheral blood, especially plasma, where an increase in the levels of said long non-coding RNA, is indicative of developing gastric cancer or an increased probability of developing this type of cancer, as occurs in patients with advanced precursor lesions (OLGA III and IV)

The invention thus provides a new biomarker and new methods for the early diagnosis of gastric cancer characterized by being non-invasive, high precision, fast in the delivery of results and low in purchasing and operating costs.

The method for determining the amount and/or concentration of the IncRNA KCNQ1OT1 in peripheral blood, especially plasma, can be by any method available in the art for RNA quantification. Preferably, the quantitative reverse transcriptase polymerase chain reaction (qRT-PCR) technique is used, but any available method that allows for RNA quantification can be used.

For example, variants of the qRT-PCR technique can be used, such as adding specific probes, like a taqman probe.

All embodiments that determine the amount and/or concentration of IncRNA KCNQ1OT1 in peripheral blood and correlate it with the early detection of gastric cancer or with the determination of the increased risk of developing gastric cancer are within the scope of the present invention.

In an especially preferred embodiment, the concentration of IncRNA KCNQ1OT1 can be evaluated by the qRT PCR technique using the primers of the invention, which have the following sequence:

Sense primer:
(SEQ ID No. 1)
5′-TGCAGAAGACAGGACACTGG-3′
Antisense primer:
(SEQ ID No. 2)
5′ CTTTGGTGGGAAAGGACAGA-3′

However, it will be obvious to the person skilled in the art that IncRNA KCNQ1OT1 is a long RNA, therefore different pairs of primers can be designed for its detection. Where the invention lies not in the pair of primers used, but in the correlation defined by the inventors between the increase in the amount and/or concentration of IncRNA KCNQ1OT1 in peripheral blood and the presence of gastric cancer or risk of gastric cancer in the individual for whom the diagnosis is made, where all possible embodiments are within the scope of the present invention.

A preferred embodiment of the invention is described below, without limiting the technical variants that a person skilled in the art may incorporate or modify, and which remain within the scope of the inventive concept that is protected in this document.

### EXAMPLES

### Example 1. Expression of KCNQ1OT1 in 3 Gastric Cancer Cell Lines

As a first step, the expression profile of KCNQ1OT1 was evaluated in 3 Gastric Cancer cell lines (in vitro) SNU5, SNU1 and Hs746T and the normal gastric cell line GES-1, as a control. For this, total RNA was extracted from each cell line, cDNA was generated and amplified by qRT-PCR using the SYBERGREEN DNA intercalator and the specific primers for KCNQ1OT1:

Sense primer:
(SEQ ID No. 1)
5′-TGCAGAAGACAGGACACTGG-3′
Antisense primer:
(SEQ ID No. 2)
5′ CTTTGGTGGGAAAGGACAGA-3′

The primer annealing temperature was 57° C.

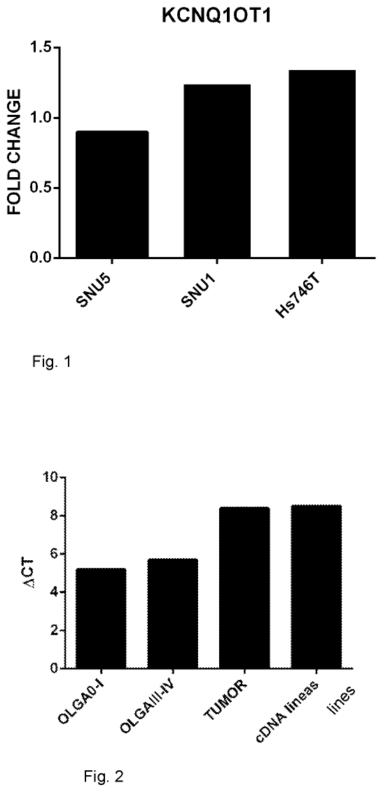

FIG. 1 shows the expression of KCNQ1OT1 in 3 gastric cancer cell lines (SNU1, SNU5 and Hs746T). The quantification of KCNQ1OT1 was obtained from the difference in Ct of this gene vs the constitutive one (Rps13). This last value was divided (normalized) by the value obtained in the normal gastric cell line GES-1.

As shown in FIG. 1, all gastric cancer cell lines evaluated have expression of KCNQ1OT1.

### Example 2. Expression of KCNQ1OT1 at Different Stages of Gastric Carcinogenesis

Once it was identified that KCNQ1OT1 is expressed in gastric cancer cell lines, we wanted to determine if this IncRNA is present in gastric tissues of different origins: low (OLGA 0-I) and high-grade (OLGA III-IV) precursor lesions, cases of gastric cancer and cell lines, the latter considered as a positive control of expression of KCNQ1OT1. Each tissue was extracted to obtain RNA, using the qiagen miRNeasy Mini Kit, this RNA was used as a template to generate cDNA. In order to obtain an overview of each type of lesion, we generated a pool of cDNAs from each type of tissue, that is: low-grade (5 samples) and high-grade (13 samples) precursor lesions, gastric cancer (4 cases) and cell lines (6 lines). Finally, the qRT-PCR was performed, as detailed above.

The results obtained are shown in FIG. 2, where it is observed that as the severity of the gastric injury increases, the expression of KCNQ1OT1 increases, further reaffirming that our in vitro model (cell lines) represents tumor samples.

### Example 3. Expression of KCNQ1OT1 in Plasma Samples

Finding that KCNQ1OT1 is expressed in gastric tissue, and that this expression is higher in tumor tissue, we wonder if this increase will be reflected in plasma samples from individuals with low-grade lesions (OLGA 0) vs. gastric cancer patients. For this, we used plasma from 3 individuals with OLGA 0 and 4 patients with gastric cancer. Five hundred μl of plasma were used to obtain RNA, using the Trizol reagent, from which cDNA was generated, which was used as a template for the qRT-PCR, explained in example 1.

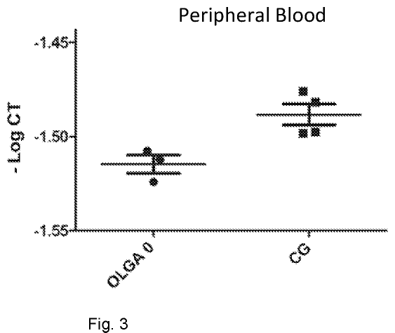

The results are shown in FIG. 3, where it is observed that the plasma of individuals with low-grade lesions (OLGA 0) has a lower expression of KCNQ1OT1 compared to the plasma of patients with gastric cancer (GC). These results show that the overexpression of KCNQ1OT1 in gastric cancer can be detected in circulating plasma, demonstrating the usefulness of KCNQ1OT1 as a biomarker for gastric cancer.

1. Method for early detection of gastric cancer or precursor lesions of gastric cancer CHARACTERIZED in that it comprises detecting an increase in the amount and/or concentration of long noncoding RNA (IncRNA) KCNQ1OT1 in peripheral blood.

2. Method according to claim 1 CHARACTERIZED in that it comprises detecting the amount and/or concentration of long noncoding RNA (IncRNA) KCNQ1OT1, in plasma.

3. Method according to claim 2 CHARACTERIZED in that it comprises detecting the amount and/or concentration of long noncoding RNA (IncRNA) KCNQ1OT1, using the polymerase chain reaction with quantitative reverse transcriptase (qRT PCR), or by qRT PCR with a taqman probe.

4. Method according to claim 3 CHARACTERIZED in that it comprises detecting the amount and/or concentration of long noncoding RNA (IncRNA) KCNQ1OT1, using the quantitative reverse transcriptase polymerase chain reaction (qRT PCR) and primers with the nucleotide sequences as defined in the (SEQ ID No. 1) and (SEQ ID No. 2).


SEQUENCE LISTING (2 sequences)

SEQ ID NO: 1 - DNA, 20 bp, Homo sapiens
tgcagaagacaggacactgg

SEQ ID NO: 2 - DNA, 20 bp, Homo sapiens
ctttggtgggaaaggacaga